(19) United States
(12) Patent Application Publication
(10) Pub. No.: US 2015/0133984 A1
(43) Pub. Date: May 14, 2015

(54) HAND TOOL ATTACHMENT ASSEMBLY

(71) Applicant: Smith & Nephew, Inc., Memphis, TN (US)

(72) Inventor: Brian Loreth, Braintree, MA (US)

(21) Appl. No.: 14/533,488

(22) Filed: Nov. 5, 2014

Related U.S. Application Data
(60) Provisional application No. 61/904,137, filed on Nov. 14, 2013.

(51) Int. Cl. A61B 17/32

(52) U.S. Cl. CPC A61B 17/32002; A61B 2017/320032; A61B 2017/00473
USPC 606/170; 29/428

(57) ABSTRACT

According to one configuration, a hand tool attachment assembly includes a cap, an outer tube, and an inner tube. The cap includes a hollowed volume to matably secure the cap to a distal end of a hand tool. The outer tube is affixed to the cap opposite the hollowed region. The inner tube is disposed in the outer tube and rotates with respect to the outer tube. A fastening resource disposed on an inner surface of the cap enables securedly attaching of the cap to the distal end of the hand tool.

### RELATED APPLICATIONS

This application is related to and claims the benefit of earlier filed U.S. Provisional Patent Application Ser. No. 61/904,137 entitled “HAND TOOL ATTACHMENT ASSEMBLY,” (Attorney Docket No. SNI13-62(PT-4293-US-PSP)p), filed on Nov. 14, 2013, the entire teachings of which are incorporated herein by this reference.

This application is related to United States patent application entitled “HAND TOOL WITH REMOVABLE ATTACHMENT ASSEMBLY,” (Attorney Docket No. SNI13-61(PT-4294-US-NP), filed on the same day as this application, the entire teachings of which are incorporated herein by this reference.

### BACKGROUND

One type of conventional arthroscopic cutting tool includes a rigid, stationary outer tube within which a rigid inner tube rotates to perform a cutting operation. A cutting implement, such as a blade or abrading burr, is disposed on the distal end of the inner tube. As the inner tube rotates within the outer tube, a tip (at a distal end) of the cutting implement is applied to a surgical site. Matter such as tissue or bone fragments cut by the rotating blade at the cutting tip are drawn through the interior of the inner tube along with irrigating fluid via suction applied to the inner tube.

An example of a conventional cutting drive assembly 200 for use in surgical applications is shown in FIG. 1. In general, to use the cutter drive assembly 200, a proximal end of the cutter drive assembly 200 is engaged into a motorized hand tool. During operation, the motorized hand tool rotates the inner tube within the outer tube to provide cutting capability at cutting tip 220. Details of an example conventional surgical instrument using cutter assembly 200 are further discussed in U.S. Pat. No. 5,601,583 issued to Donahue, et al.

### BRIEF DESCRIPTION

A drawback of the conventional cutter tool as shown in FIG. 1 is that the complex and expensive-to-manufacture drive portion at proximal end of assembly 200 is thrown away with the cutter blade portion after use. Thus, use of assembly 200 to perform cutting operations can be costly.

In contrast to conventional cutter tools, embodiments herein include a blade assembly (a.k.a., a hand tool attachment) configured to include a cap, an outer tube, and an inner tube. The cap of the blade assembly includes a hollowed volume to matably secure the cap (and corresponding blade assembly) to a distal end of a hand tool. The outer tube is affixed to the cap. The inner tube is disposed in the outer tube and rotates within the outer tube. A proximal end portion of the inner tube extends into and resides in the hollowed volume of the cap and couples to a drive mechanism when the blade assembly is attached to the hand tool. In one embodiment, the proximal end portion of the inner tube includes a keyed interface for coupling to the drive mechanism of the hand tool.

The cap of the blade assembly can be configured to include one or more fastening resources to fasten the cap of the assembly to a corresponding hand tool. For example, in one embodiment, the one or more fastening resources includes protrusions, threads, etc., disposed on an inner surface of the cap to securedly attach the cap to the distal end of the hand tool.

In accordance with further embodiments, a distal end of the inner tube includes a window and respective blade to cut material such as tissue. For example, a distal end of the outer tube includes an opening such as a cutting window. The blade of the inner tube is disposed to rotate in a portion of the outer tube including the cutting window.

By further way of non-limiting example embodiment, the inner tube is longer than the outer tube such that a proximal end of the inner tube extends into the hollowed volume of the cap as previously discussed. The proximal end of the inner tube includes a keyed interface for coupling to a distal end of a drive adapter in a cavity of the hand tool.

If desired, when the blade assembly is removed from the hand tool, the inner tube can be slidably removed from the outer tube to support operations such as replacement, cleaning, etc.

Additional embodiments herein include fabricating a respective blade assembly. For example, in one embodiment, a fabricator resource receives a first component such as a cap. The cap is configured to include a hollowed volume to matably secure the cap to a distal end of a respective hand tool. The fabricator further receives a second component such as a first tube (outer tube). The first tube includes a cutting window disposed at a respective distal. The fabricator resource affixes a proximal end of the first tube to a facing of the cap opposite the hollowed volume. A distal end of the first tube includes a cutting window. The fabricator resource further receives a second tube. The fabricator resource inserts the second tube (inner tube) into the first tube. The second tube is fabricated to include a blade that resides in the corresponding cutting window of the first tube. The blade of the second tube is rotatable in the first tube. If desired, the distal tip of the first tube can include a stop to prevent the second tube from sliding beyond the cutting window. As previously discussed, the second tube (inner tube) is slidably removable from the first tube (outer tube think it) for easy cleaning and/or replacement.

Further embodiments herein include use of the hand tool and corresponding blade assembly to perform a cutting operation. In one embodiment, a user receives or obtains a hand tool. A distal end of the hand tool opposite a handle of the hand tool includes an opening and a hollowed volume. A rotatable drive adapter resides in a hollowed volume of the hand tool. The user receives or obtains a blade assembly. As previously discussed, the blade assembly includes an inner tube disposed in an outer tube extending axially from a cap of the blade assembly.

The user engages an end of the inner tube passing through the cap into the rotatable drive adapter of the hand tool. The user then secures the cap of the blade assembly to the distal end of the hand tool. In one embodiment, securing of the cap blade assembly to the distal end of the hand tool causes the hollowed volume or cavity to become a fluid-tight chamber in which the rotatable drive adapter resides. In other words, the cap of the blade assembly covers the opening of the opening at the distal end of the hand tool to create the fluid-tight chamber.

In accordance with further embodiments, the rotatable drive adapter located in the hollowed volume of the hand tool is spring-loaded. Securing the cap of the blade assembly to the distal end of the hand tool causes the spring-loaded rotatable drive adapter to exert an axial force on the inner tube of the blade assembly towards a cutting window on a distal end of the outer tube. As previously discussed, the distal end of the outer tube can be configured to include a stop, preventing the inner tube from sliding beyond the cutting window.

Subsequent to attaching the blade assembly to the distal end of the hand tool, the user operates a drive control resource of the hand tool to rotate the rotatable drive adapter. Activation of the rotatable drive adapter causes the inner tube of the blade assembly to rotate inside of the outer tube.

If further desired, the user can open a respective valve to apply suction to the fluid-tight chamber. Application of the suction to the fluid-tight chamber draws any matter passing through the cutting window at the tip of the blade assembly through the inner tube and a hollowed volume of the rotatable drive adapter out a respective suction port of the fluid-tight chamber.

Subsequent to engaging and securing the blade assembly to the distal end of the hand tool, and after completing a respective procedure, the user: i) removes the cap from the distal end of the hand tool, and ii) exerts a pulling force on the inner tube to remove the inner tube from the outer tube. As previously discussed, the inner tube can be cleaned for subsequent use or replaced with a new inner tube.

Embodiments herein are useful over conventional surgical instruments for a number of reasons. For example, the tool assembly as described herein is substantially lower in complexity than conventional cutter blade assemblies. Lower complexity results in benefits such as lower fabrication costs, ease of attaching the blade assembly to a corresponding hand tool, etc. These and other more specific embodiments are disclosed in more detail below.

As discussed herein, techniques herein are well suited for use in cutting applications. However, it should be noted that embodiments herein are not limited to use in such applications and that the techniques discussed herein are well suited for other applications as well.

Additionally, note that although each of the different features, techniques, configurations, etc., herein may be discussed in different places of this disclosure, it is intended, where suitable, that each of the concepts can optionally be executed independently of each other or in combination with each other. Accordingly, the one or more present inventions as described herein can be embodied and viewed in many different ways.

Also, note that this preliminary discussion of embodiments herein purposefully does not specify every embodiment and/or incrementally novel aspect of the present disclosure or claimed invention(s). Instead, this brief description only presents general embodiments and corresponding points of novelty over conventional techniques. For additional summary and details and/or possible perspectives (permutations) of the invention(s), the reader is directed to the Detailed Description section and corresponding figures of the present disclosure as further discussed below.

### BRIEF DESCRIPTION OF THE DRAWINGS

The foregoing and other objects, features, and advantages of the invention will be apparent from the following more particular description of preferred embodiments herein, as illustrated in the accompanying drawings in which like reference characters refer to the same parts throughout the different views. The drawings are not necessarily to scale, with emphasis instead being placed upon illustrating the embodiments, principles, concepts, etc.

FIG. 1 is an example perspective image of a cutter assembly according to conventional techniques.

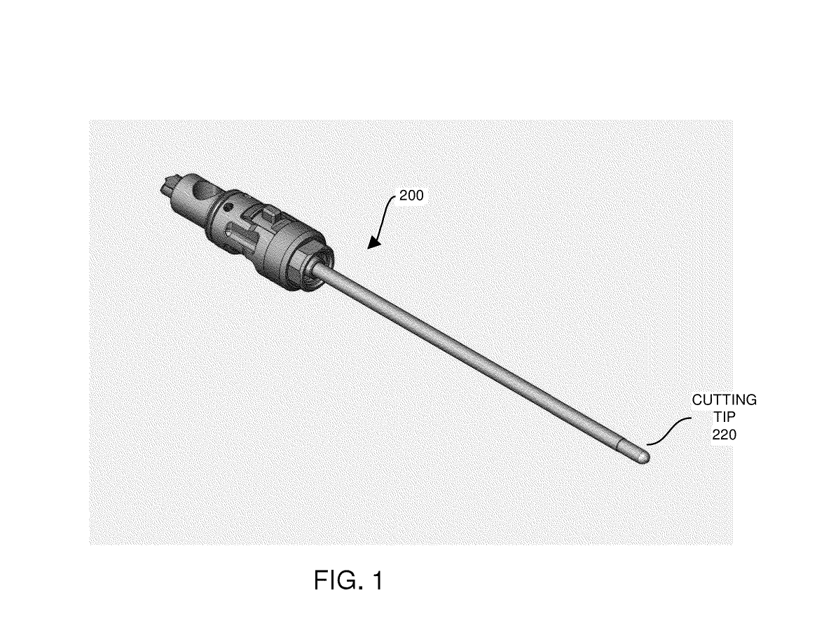

FIG. 2 is an example perspective view diagram of a cutter blade assembly selectively coupled to a hand tool according to embodiments herein.

FIG. 3 is an example side view diagram of a distal end of a cutter blade assembly according to embodiments herein.

FIG. 4 is an example side view diagram of a cutter blade assembly coupled to a drive unit according to embodiments herein.

FIG. 5 is an example perspective view diagram of a cutter blade assembly coupled to a drive unit according to embodiments herein.

FIG. 6 is an example cutaway side view diagram of a hand tool and corresponding drive unit according to embodiments herein.

FIG. 7 is an example cutaway perspective view diagram of a driver adapter according to embodiments herein.

FIG. 8 is an example perspective view diagram of a drive adapter according to embodiments herein.

FIG. 9 is an example diagram illustrating spring loading of a driver adapter according to embodiments herein.

FIG. 10 is an example perspective diagram of a cutter system according to embodiments herein.

FIG. 11 is a method of fabricating a blade assembly according to embodiments herein.

FIG. 12 is a method of using a hand tool and respective blade assembly according to embodiments herein.

FIG. 13 is an example cutaway perspective view diagram illustrating use of a respective retainer resource to secure a drive adapter to a driveshaft assembly according to embodiments herein.

### DETAILED DESCRIPTION

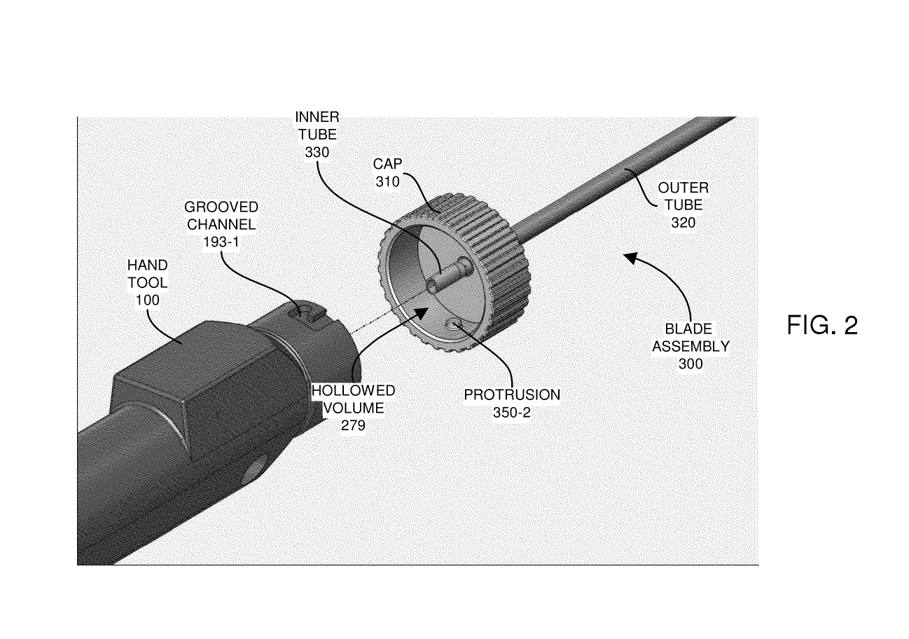

Now, more specifically, FIG. 2 is an example perspective diagram illustrating a proposed blade assembly 300 according to embodiments herein. As shown, blade assembly 300 includes cap 310, inner tube 330, and outer tube 320.

Cap 310 includes hollowed volume 279 for coupling the blade assembly 300 to the distal end of hand tool 100.

If desired, cap 310 can include any suitable type of fastener resource such as grooved regions, threads, etc., facilitating twisting and securing of the cap 310 to a respective distal end of hand tool 100. In one non-limiting example embodiment, the hand tool 100 includes one or more channels including grooved channel 193-1. Cap 310 includes one or more protrusions such as protrusion 350-2 that secure cap 310 to the distal end of hand tool 100. As further discussed below, use of protrusions and grooved channels to secure the 310 to the distal end of hand tool 100 is shown by way of non-limiting example only. Any suitable technique can be used to secure cap 310 to the distal end of hand tool 100.

Note that each of the components in FIG. 2 such as cap 310, inner tube 330, and outer tube 320 can be fabricated from any suitable type of material such as metal, plastic, etc.

In one embodiment, inner tube 330 slides and rotates within outer tube 320 and is removable. Inner tube 330 is made of substantially rigid material or semi-rigid material and extends at least a length of outer tube 320. A proximal end (neuron) of outer tube 320 is fixedly attached to cap 310.

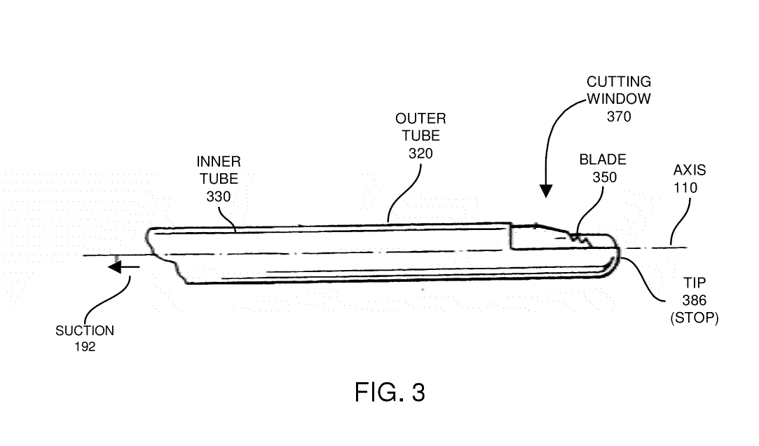

As further shown in FIG. 3, by further way of a non-limiting example, distal end of outer tube 320 includes a respective cutting window 370. In one embodiment, distal end of the inner tube 330 includes a respective blade 350 (potentially with serrated teeth, razor-edge, etc.) and corresponding cutting window that cuts tissue residing within cutting window 370 of outer tube 320.

During operation, hand tool 100 controls application of suction 192 to inner tube 330. Suction 192 applied to inner tube 330 causes loose matter in a vicinity of the spinning blade 350 at distal end of inner tube 330 to be drawn through cutting window 370 at the distal end of tube 320 and respective opening (a rotating second cutting window) at the distal end of the inner tube 330 towards hand tool 100.

Referring again to FIG. 2, in this non-limiting example embodiment, cap 310 selectively connects to distal end of hand tool 100 including one or more grooved channels 193. For example, one or more protrusions 350 such as protrusion 350-2 on inside circumferential surface of cap 310 slides into a respective grooved channel 193-2. After sliding, a twist of cap 310 secures the cap 310 to distal end of hand tool 100.

Note that as an alternative to a grooved channel/protrusion locking mechanism, any suitable type of means can be used to secure the cap 310 to the distal end of the hand tool 100. For example, as an alternative to a J-groove locking mechanism, an outer circumference of the distal end of the hand tool 100 can be configured to include threads. Corresponding threads located on the inner circumferential wall of cap 310 can be configured to accept threads on the inner circumferential surface of the cap 310. In such an instance, the cap 310 can be screwed onto the distal end of hand tool 100.

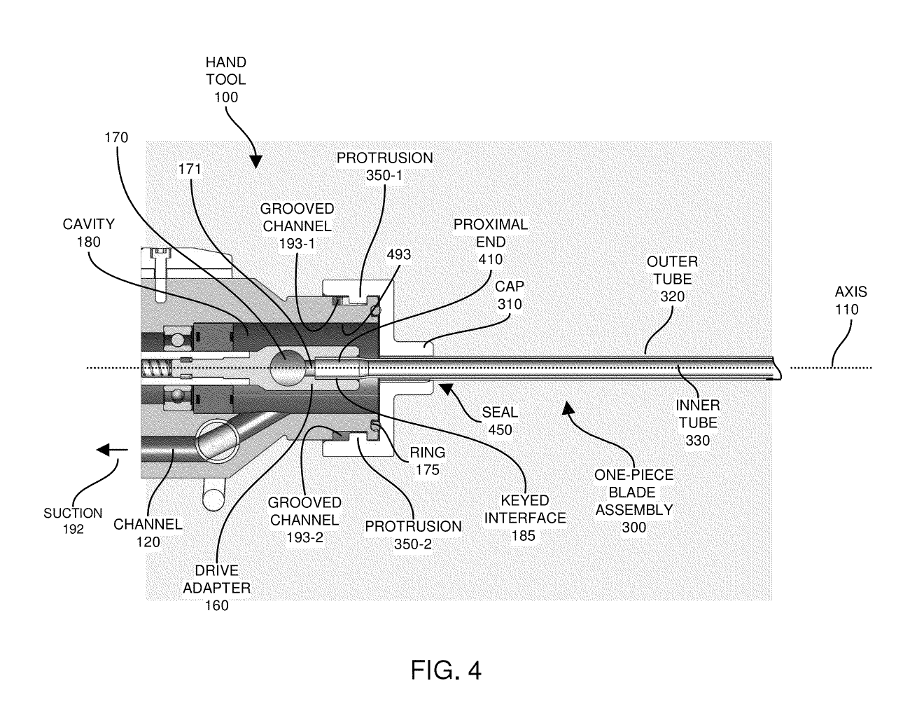

FIG. 4 is an example cross-sectional view diagram illustrating a proposed one-piece blade adapter attached to a corresponding hand tool according to embodiments herein.

As shown, and as previously discussed, cap 310 couples to distal end of hand tool 100. Protrusions 350 (such as protrusions 350-1, 350-2, etc.) slide into respective grooved channels 193 (grooved channel 193-1, grooved channel 193-2, etc.) to lock cap 310 in place onto distal end of hand tool 100. More specifically, protrusion 350-1 slides in grooved channel 193-1; protrusion 350-2 slides in grooved channel 193-2; etc.

Subsequent to rotating the cap 310 to secure cap 310 to the distal end of the hand tool 100, ring 175 and seal 450. The seal 450 can be created based on a fluid-tight fit (such as via overmolding, heat staking, etc.) between outer circumferential surface of the outer tube 320 and respective inner circumferential surface of an opening disposed in cap 310 as shown. If desired, seal 450 can include material such as glue, silicone, etc.

Seal 450 ensures that cavity 180 is fluid-tight with respect to surrounding environment outside of cavity 180. As will be discussed in more detail later in the specification, suction 192 applied to channel 120 and respective cavity 180 causes matter received in a respective window at distal end of outer tube 320 to flow through a path including: inner tube 330, proximal end 410, keyed interface 185, hollowed volume 171, hollowed volume 170, into and through cavity 180 to channel 120.

During operation, the distal end of drive adapter 160 rotates about respective axis 110. The outer surface of the drive adapter 160 is free from contacting corresponding outer circumferential surface 493 in the cavity 180. In other words, the outer surface of the drive adapter 160 does not contact the surface 493 in cavity 180 such that the drive adapter 160 is free to rotate without friction.

Further, proximal end 410 of inner tube 330 matably attaches to keyed interface 185. Thus, rotation of the drive assembly 160 about axis 110 causes the inner tube 330 to rotate about axis 110. As previously discussed, inner tube 330 rotates with respect to outer tube 320.

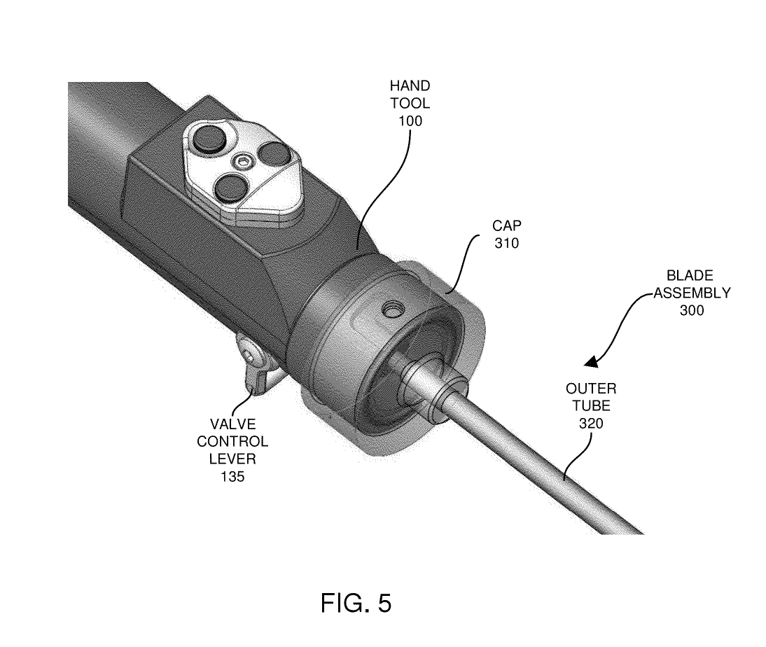

FIG. 5 is an example perspective diagram illustrating attachment of the blade assembly 300 to the distal end of hand tool 100 according to embodiments herein. As further discussed below, valve control lever 135 controls an amount of suction 195 applied to the inner tube 330.

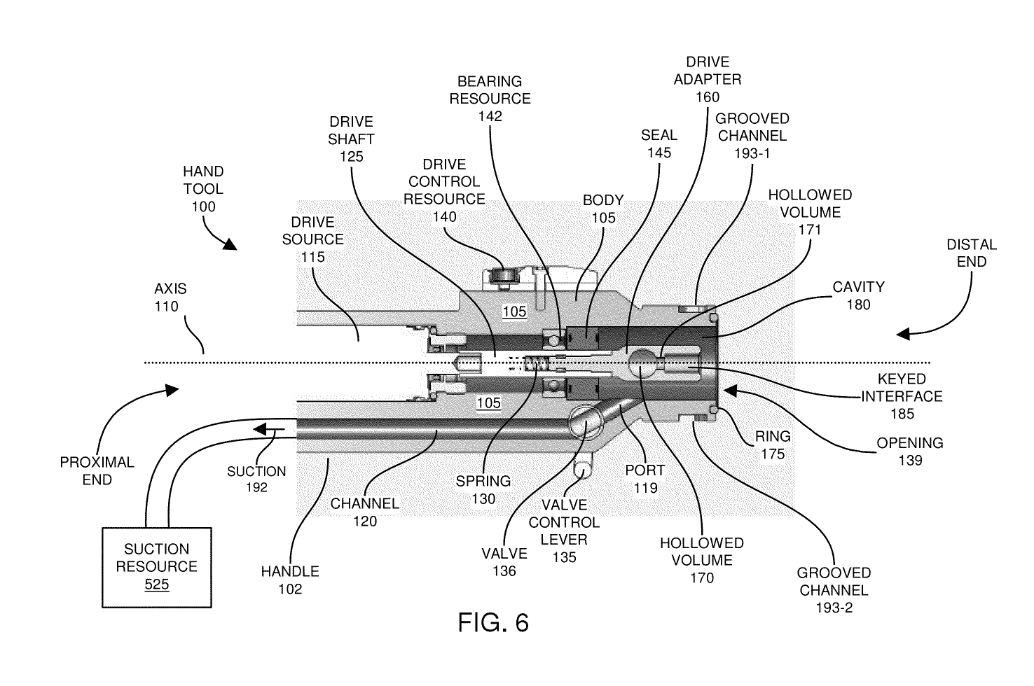

FIG. 6 is an example diagram illustrating a more detailed version of a proposed drive source according to embodiments herein.

As shown, hand tool 100 includes a handle portion 102 disposed at the proximal end of hand tool 100 for gripping by a respective user such as a surgeon. When the respective cap 310 of the assembly element 300 is securely attached to the distal end of hand tool 100, the user gripping handle portion 102 of hand tool 100 is able to steer the corresponding tip 386 (and corresponding cutting window 370) to a respective location such as a surgical site.

As further shown, drive shaft 125 (i.e., a drive component) is matably attached to drive source 115 in hand tool 100. Activation of the drive source 115 (such as a spinning electric motor, pneumatic motor, etc.) causes rotation of drive shaft 125 about axis 110.

Note that the drive shaft 125 can be fabricated from any suitable one or more types of material such as hardened stainless steel, plastic, epoxy, etc.

Bearing resource 142 disposed in body 105 of hand tool 100 facilitates rotation of drive shaft 125 with respect to (substantially stationary) body 105 of hand tool 100. Presence of seal 145 prevents debris in cavity 180 from passing to bearing resource 142.

Additionally, hand tool 100 includes drive control resource 140. The user operating hand tool 100 controls the drive control resource 140 (such as one or more buttons, triggers, etc.) to selectively activate rotation of the drive shaft 125 (i.e., a drive component).

As a specific example, pressing a respective button of drive control resource 140 to an ON position causes the drive source 115 to spin drive shaft 125 about axis 110. Releasing the button of drive control resource 140 to an OFF position causes the drive resource 115 to cease spinning of the drive shaft 125 about axis 110.

If desired, the drive control resource 140 can be configured to one or more speed buttons to control the speed of rotating the drive adapter 160 and corresponding inner tube 330.

As further shown, distal end of hand tool 100 includes cavity 180 (such as hollowed volume or housing) in which drive adapter 160 resides. Drive adapter 160 (such as made from stainless steel material or other suitable material) is mechanically coupled to a distal end of drive shaft 125 such that rotation of the drive shaft 125 (and drive source 115) causes rotation of drive assembly 160 as well. As previously discussed, the proximal end 410 of the inner tube 330 can be configured to reside in keyed interface 185 upon securing of cap 310 to the distal end of hand tool 100. Rotation of the drive shaft 125 causes rotation of drive adapter 160; rotation of drive adapter 160 causes rotation of inner tube 330.

Accordingly, rotational forces produced by drive source 115 translate through drive shaft 125 and drive adapter 160 to inner tube 330.

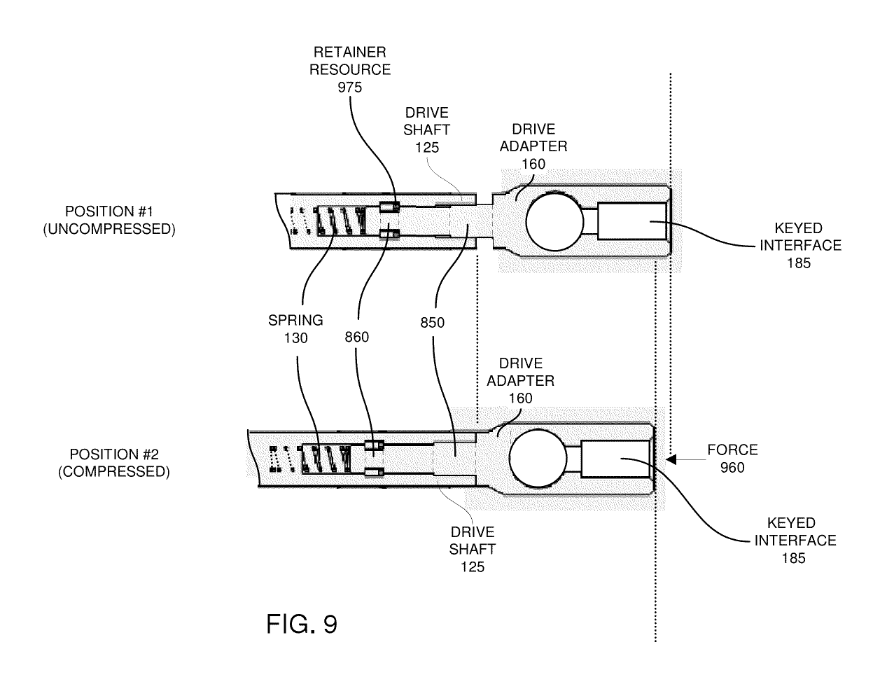

In one embodiment, a corresponding end of the drive adapter 160 (e.g., in and near the drive source 115) slidably moves in a hollowed volume (keyed-bore) of drive shaft 125 along axis 110 with respect to drive shaft 125. Thus, the drive adapter 160 slides within a respective bore (receptacle) at a distal end of the drive shaft 125. When the drive assembly 160 is pushed inward towards drive source 115 via application of a respective force substantially along axis 110, spring 130 compresses (a state as shown). When the force applied to the drive adapter 160 is removed, spring 130 decompresses and pushes drive assembly 160 outward again to a corresponding resting position in cavity 180. (This operation is further illustrated in FIG. 9 and corresponding text.)

Thus, in addition to the drive adapter 160 spinning about axis 110 along with drive shaft 125, the drive assembly 160 movably slides in cavity 180 along axis 110 depending on application of a corresponding force applied to the distal end of drive adapter 160.

Additionally, drive adapter 160 can be anchored in any suitable manner (such as via a retaining ring, clip, etc.) to the drive shaft 125 so that it does not release (or easily release) from drive shaft 125 based on application of force to the drive adapter 160 along axis 110 to pull the drive adapter 160 out of cavity 180. Further, as previously discussed, the proximal and 410 of the inner tube 330 resides in keyed interface 185 of the drive adapter 160 when the cap 310 is secured to the distal end of hand tool 100 at opening 139. Anchoring of the drive adapter 160 to the drive shaft 125 ensures that when the proximal and 410 of the inner tube 330 is removed from (pulled out of) the keyed interface 185, the drive adapter 160 is not pulled free from the drive shaft 125 out of cavity 180 (i.e., an open or closed hollowed volume depending upon whether cap 310 is secured to the distal end of the hand tool 100).

In certain instances, however, the drive adapter 160 can be configured to be removable from the distal end of drive shaft 125 and cavity 180 for maintenance.

Yet further in these non-limiting example embodiments, cavity 180 includes seal 145 (such as made from polymer material or other suitable materials as part of a sealing system or assembly) to prevent matters such as fluid, debris, etc., disposed in cavity 180 from contaminating bearing resource 142, drive source 115, etc.

For example, an outer circumferential surface of seal 145 contacts a corresponding inner circumferential surface of cavity 180 disposed in body 105 of hand tool 100; an inner circumferential surface of seal 145 contacts a corresponding outer surface of drive shaft 125. There is relatively low friction between the inner compass circumferential surface of seal 145 and the corresponding outer circumferential surface of drive shaft 125. The tightness of fitting seal 145 with respect to body 105 and drive shaft 125 enables unimpeded rotation of drive shaft 125 and corresponding drive adapter 160, but prevents contaminants in cavity 180 from passing through seal 145 to bearing resource 142, drive source 115, etc.

Thus, via seal 145, drive source 115 and bearing resource 142 are not exposed to contaminants that may be presence or passing through cavity 180.

Channel 120 (such as a tubular region or lumen) extends from port 119 of cavity 180 disposed at distal end of hand tool 100 to a proximal end of hand tool 100 where suction 192 is applied via activation of suction resource 525. In other words, suction resource 525 can be configured to selectively produce a vacuum or negative pressure, resulting in suction 192 of matter through channel 120 towards suction resource 525.

In this non-limiting example embodiment, the valve control lever 135 controls a position of corresponding valve 136 and corresponding flow from cavity 180 through channel 120 to suction resource 525. For example, opening of valve 136 enables suction 192 generated by suction resource 525 to pull matter (such as solids, liquids, and gases) in cavity 180 through port 119 into and through channel 120.

Note further that a distal end of drive assembly 160 includes hollowed volume 170 (such as a radial bore, an axis of which is disposed orthogonal or substantially orthogonal with respect to axis 110). Hollowed volume 171 (an axial bore) extends along axis 110 from distal end of the drive assembly 160 to hollowed volume 170.

The combination of hollowed volume 170 and hollowed volume 171 provides a hollowed passageway extending from an opening (such as keyed interface 185) at distal end of drive adapter 160 into cavity 180. As previously discussed, when suction 192 is applied to channel 120 when valve 136 is OPEN, suction 192 through channel 120 and port 119 provides passage of matter in cavity 180 to proximal end of hand tool 100. Thus, port 119 enables application of suction to cavity 180.

Referring again to FIG. 4, the keyed interface 185 at distal end of drive adapter 160 accepts proximal end 410 of inner tube 330 associated with blade assembly 300. The keyed interface 185 ensures that the corresponding inner tube 330 rotates about axis 110 when the corresponding drive source 115 is activated via drive control resource 140. As previously discussed, rotation or spinning of the inner tube 330 about axis 110 (in accordance with drive source 115) enables distal end of inner tube 330 exposed in cutting window 370 to cut matter such as tissue.

Because the inside of inner tube 330 is hollow, the suction 192 applied to cavity 180 causes matter (such as a solid, liquid, or gas material) passing through cutting window 370 to be sucked through a fluid pathway including inner tube 330, hollowed volume 171, and hollowed volume 170 to cavity 180 (such as a fluid-tight chamber). As previously discussed, matter in cavity 180 is sucked through channel 120 to suction resource 525 via application of suction 192.

Further note that a distal end of the hand tool 100 can include a sealing resource such as a ring 175 (fabricated from rubber or other suitable elastic material) and corresponding grooved channels 193 (such as grooved channel 193-1 and grooved channel 193-2) to lock and seal a respective cap portion of the rotatable blade assembly to the distal end of hand tool 100. Inclusion of the ring 175 ensures that the cavity 180 becomes a fluid-tight when the respective cap 310 is coupled to cover opening 139 at the distal end of hand tool 100. That is, ring 175 prevents leakage of air into cavity 180 through respective sidewalls of capacitor 310 and a respective outer surface of distal end of the hand tool 100. As previously discussed, attachment of the cap 310 to the distal end of hand tool 100 can be achieved in any suitable manner. For example, the cap 310 can be secured to the distal end of hand tool 100 via threads, J-locks, etc.

As an alternative to including ring 175 in a groove at the distal end of the hand tool 100, embodiments herein can further include disposing a respective a seal resource (such as a gasket. O-ring, etc.) on a facing of the cap 310 in the hollowed volume 279. Presence of the gasket in cap 310 ensures that the cavity 180 is fluid-tight.

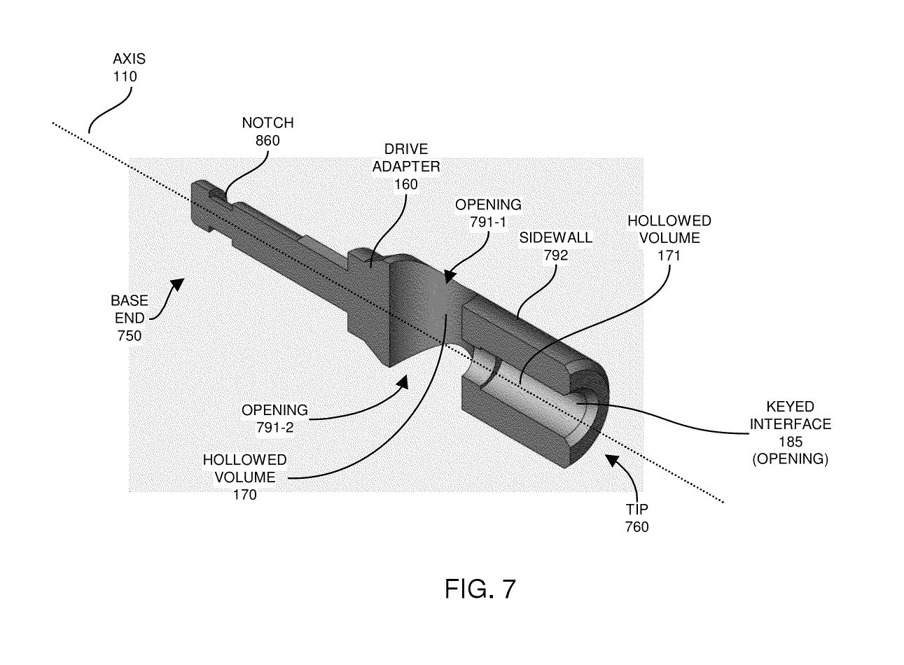

FIG. 7 is an example cutaway perspective view diagram of a driver adapter according to embodiments herein.

As shown in this cutaway perspective view diagram, the drive adapter 160 includes hollowed volume 170 and hollowed volume 171. By way of non-limiting example embodiment, the hollowed volume 171 of drive adapter 160 includes keyed interface 185 (such as an opening) at tip 760, which accepts proximal end 410 of the inner tube 330.

Alternatively, note that the keyed interface 185 can be disposed on an outer circumferential surface at a tip of the drive adapter 160 as opposed to being a cavity or opening at the distal end of the drive adapter. In such an instance, the keyed interface 185 disposed on the outer surface of the drive adapter 160 fits into a matching keyed opening at the proximal end 410 of the inner tube 330. In other words, in this latter embodiment, the opening at the proximal and 410 of the inner tube 330 acceptably receives the keyed interface 185 disposed at the distal end of drive adapter 160.

Accordingly, the keyed interface 185 can be located on the internal or external surface of the drive adapter 160 depending on the embodiment.

As previously discussed, inclusion of the keyed interface 185 ensures that the inner tube 330 and drive adapter 160 rotate in unison about axis 110 when the drive adapter 160 is rotated about axis 110.

As further shown, the hollowed volume 170 in the drive adapter extends to the sidewall 792 of the drive adapter 180, creating openings 791-1 and opening 791-2.

As further discussed in the following FIGS., the base end 750 of the drive adapter 180 includes notch 860 to accept a retainer resource such as a retaining ring, spring clip, etc., preventing the drive adapter 160 from being pulled out of the drive shaft 125 and corresponding cavity 108.

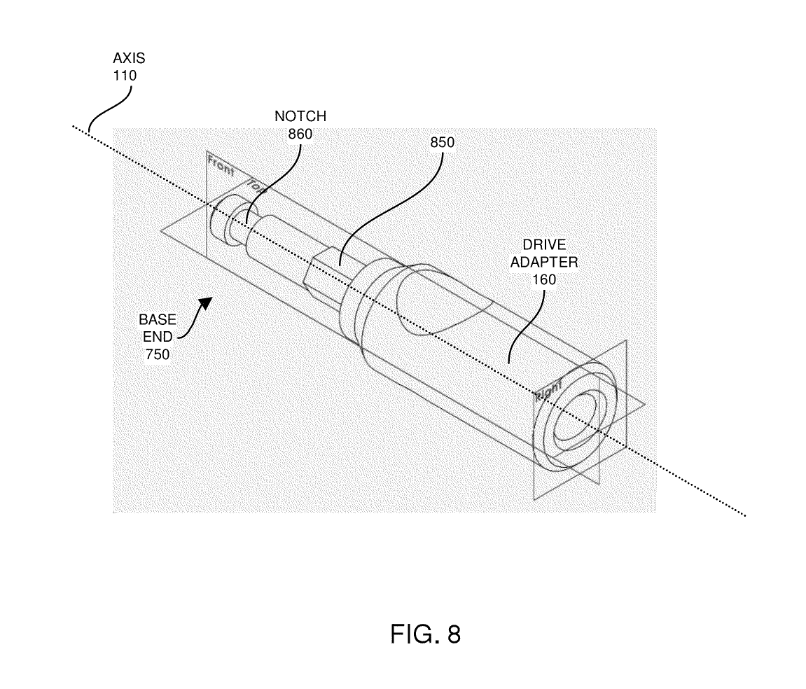

FIG. 8 is an example perspective view diagram of a drive adapter according to embodiments herein.

As shown in this example embodiment, drive adapter 160 includes keyed region 850 (such as a hexagonal bolt pattern or other suitable pattern) that fits into a corresponding hollowed portion at a distal end of drive shaft 125 as previously discussed. The keyed (but axially slidable) fit between the drive shaft 125 and keyed region 850 of the drive adapter 160 ensures that the drive shaft 125 and drive adapter 160 spin together about axis 110 when a corresponding rotational force is applied to the drive shaft 125.

Additionally, drive adapter 160 includes one or more a holding mechanism such as a notch 860 to accept the retaining ring or clip to secure or anchor the driver adapter 160 within drive shaft 125. In one embodiment, the anchor enables the base end 750 of the drive adapter 160 to slide within a corresponding cavity of the drive shaft 125. Anchoring or securing of the drive adapter 160 to the drive shaft 125 ensures that the drive adapter 160 is not easily pulled free from the drive shaft 125 when the corresponding proximal end 410 of the inner tube 330 is removed from the keyed interface 185 of the drive adapter 160.

In accordance with further embodiments, the drive adapter 160 is removable from the cavity 180 to facilitate cleaning of matter from the cavity 180.

In contrast to conventional techniques, the drive adapter 160 can be reused to perform cutting operations because it is not disposed of with a corresponding blade assembly.

FIG. 9 is an example diagram illustrating spring loading of a driver adapter according to embodiments herein.

As shown, in an uncompressed position #1, the spring 130 applies a force to drive adapter 160, pushing drive adapter 160 to an extended position. One or more anchors (such as retainer resource 975) in notch resource 860 prevent the drive assembly 160 from being pulled away from driveshaft 125.

As further shown in position #2, application of force 960 to the drive adapter 160 causes the spring 130 to compress and movement of the drive assembly 160 into driveshaft 125.

Whether in the compressed or uncompressed position, the insertion of keyed region 850 (slidable within the driveshaft 125 as previously discussed) into a corresponding cavity of the driveshaft 125 ensures that the drive adapter 160 rotates along with driveshaft 125 when drive source 115 applies a corresponding torque to driveshaft 125.

By way of non-limiting example embodiment, the spring 130 can be configured to exert a force 960 between 0.1 and 5 pounds to the drive adapter 160. However, note that the spring 130 (or other suitable compressible resource) can be configured to apply any suitable compression force to drive adapter 160. Thus, the amount of force 960 needed to compress the spring 130 can vary depending upon the embodiment.

As previously discussed, the force 960 provided by spring 130 ensures that the blade 350 at the distal end of inner tube 330 is pushed up against tip 386 disposed on the distal end of outer tube 320.

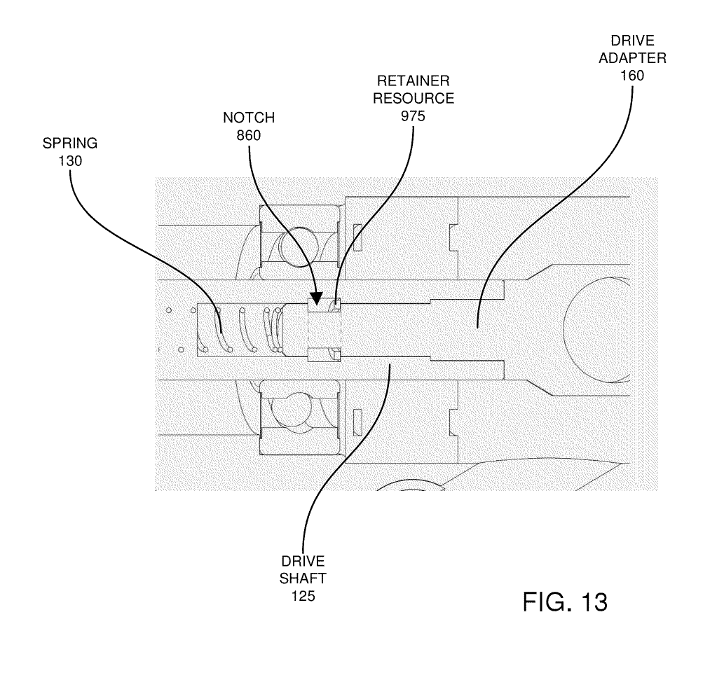

FIG. 13 is an example cutaway perspective view diagram illustrating use of a respective retainer resource to securely attach a drive adapter to a driveshaft assembly according to embodiments herein.

Specifically, as shown, retainer resource 975 disposed in notch 860 secures the drive adapter 160 to the drive shaft 125. As previously discussed, the retainer resource 975 enables the notched end of drive adapter 160 (i.e., an end of the drive adapter 160 including notch 860) to slide within a respective cavity of the drive shaft 125.

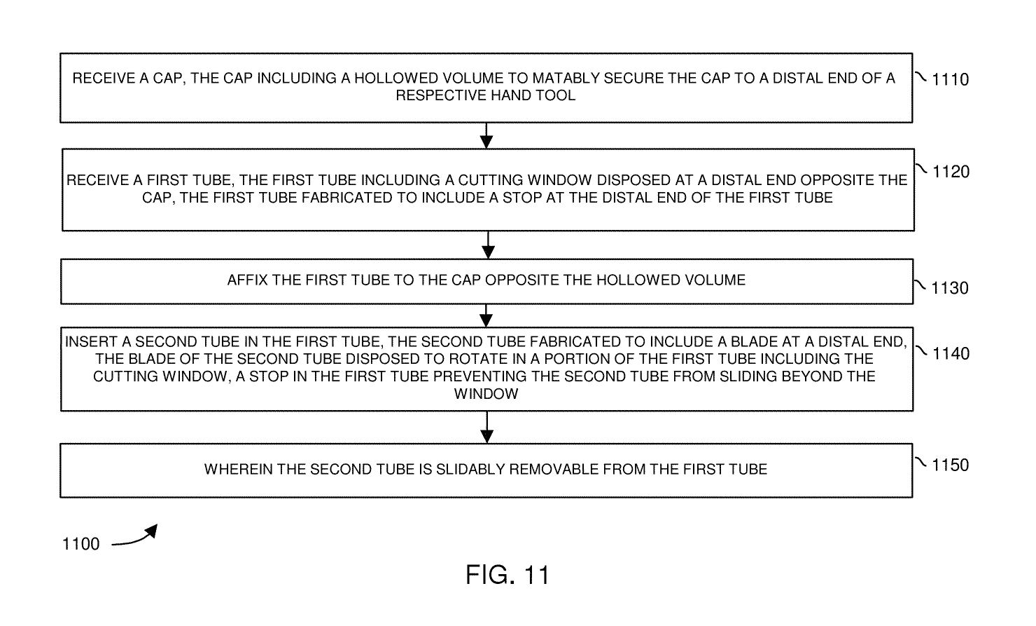

FIG. 11 is a flowchart 1100 illustrating an example method according to embodiments. Note that there will be some overlap with respect to concepts as discussed above.

In processing block 1110, a fabrication resource (such as a manufacturing facility, assembly-line worker, automated assembler, etc.) receives a cap. The cap includes a hollowed volume 279 to matably secure the cap 310 to a distal end of a respective hand tool 100.

In processing block 1120, the fabrication resource receives a first tube. The first tube (outer tube 320) includes a cutting window 370 disposed at a distal end opposite the cap 310, the first tube fabricated to include a stop at the distal end (tip 386) of the first tube.

In processing block 1130, the fabrication resource affixes the first tube to a facing of the cap 310 opposite the hollowed volume 279.

In processing block 1140, the fabrication resource inserts a second tube (such as inner tube 330) in the first tube, the second tube is fabricated to include a blade 350 at a respective distal end. The blade 350 of the second tube is disposed to rotate in a portion of the first tube including the cutting window 370. A tip 386 (stop) in the first tube prevents the second tube from sliding beyond the window. As previously discussed, the second tube (inner tube 330) is slidably removable from the first tube (outer tube 320).

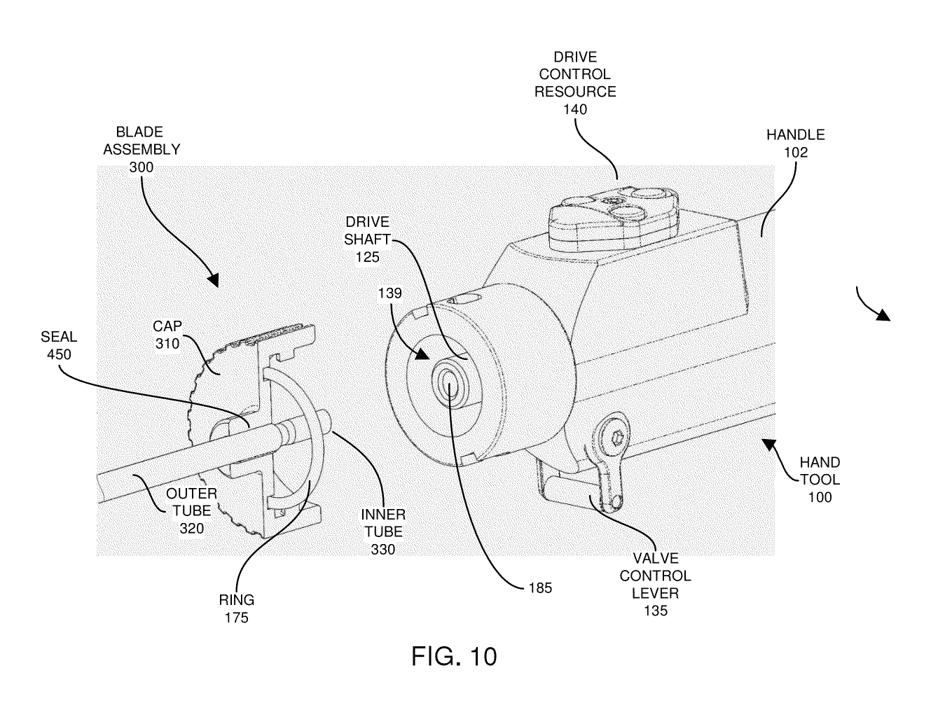

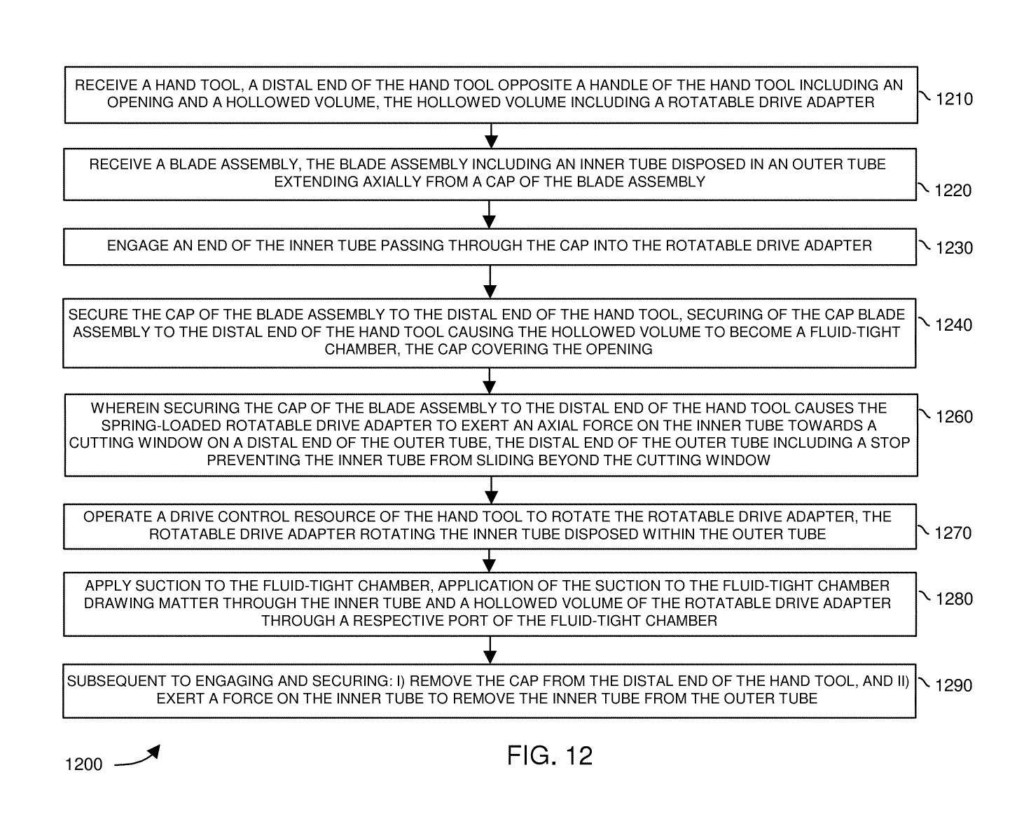

FIG. 12 is a flowchart 1200 illustrating an example method according to embodiments. Note that there will be some overlap with respect to concepts as discussed above. The flowchart 1200 will make reference to the different components of hand tool 100 and blade assembly 300 in FIG. 10.

In processing block 1210, a user receives hand tool 100; a distal end of the hand tool 100 opposite a handle 102 of the hand tool 100 includes an opening 139 and a hollowed volume (cavity 180); the hollowed volume includes a rotatable drive adapter 160.

In processing block 1220, the user receives a blade assembly 300. The blade assembly 300 includes an inner tube 330 disposed in an outer tube 320 extending axially from cap 310 of the blade assembly 300.

In processing block 1230, the user engages an end of the inner tube 330 passing through the cap 310 into keyed interface 185 of the rotatable drive adapter 160.

In processing block 1240, the user secures the cap 310 of the blade assembly 300 to the distal end of the hand tool 100 including fastening resource such as grooved channels 193 and protrusions 350. The user secures the cap 310 of blade assembly 300 to the distal end of the hand tool 100, causing the hollowed volume (cavity 180) to become a substantially fluid-tight chamber. The cap 310 covers the opening at the distal end of hand tool 100.

In processing block 1250, in one embodiment, securing of the cap 310 of the blade assembly 300 to the distal end of the hand tool 100 causes the spring-loaded rotatable drive adapter 160 to exert an axial force on the inner tube 330 away from the distal end of hand tool 100 towards a cutting window 370 on a distal end of the outer tube 320. As previously discussed, the distal end of the outer tube 320 includes a stop (such as tip 386), which prevents the blade 350 of the inner tube 330 from sliding beyond the cutting window 370. As previously discussed, the distal end of the inner tube 330 also includes an opening similar to cutting window 370 disposed on outer tube 320.

In processing block 1260, the user operates drive control resource 140 of the hand tool 100 to rotate the rotatable drive adapter 160. As previously discussed, because the outer tube 320 is secured to the cap 310 via seal 450, the rotatable drive adapter 160 rotates the inner tube 330 disposed within the outer tube 320 of assembly 300. Outer tube 320 remains stationary with respect to cap 310 and hand tool 100.

In processing block 1270, via the valve control lever 135, the user applies suction to the fluid-tight chamber formed by cap 310 and cavity 180. Application of the suction to the fluid-tight cavity 180 draws causes any matter to be drawn through the inner tube 330 and hollowed volumes 170 and 171 of the rotatable drive adapter 160 out of a respective port 119 to suction resource 525. The user uses the combination of hand tool 100 and attached blade assembly 300 to perform a cutting operation via movement of the cutting window 370 to the appropriate site.

In processing block 1280, subsequent to using the hand tool 100 and corresponding blade assembly 300 to perform a cutting operation, the user removes the cap 310 and corresponding blade assembly 300 from the distal end of the hand tool 100. The user then exerts a removal force on the proximal end of the inner tube 330 extending through the cap 310 to remove the inner tube 330 from the outer tube 320. As previously discussed, the removed inner tube 330 can be cleaned and refurbished or replaced with a new inner tube 330.

Note again that techniques herein are well suited for use in cutting, grinding, etc., types of applications. However, it should be noted that embodiments herein are not limited to use in such applications and that the techniques discussed herein are well suited for other applications as well.

Based on the description set forth herein, numerous specific details have been set forth to provide a thorough understanding of claimed subject matter. However, it will be understood by those skilled in the art that claimed subject matter may be practiced without these specific details. In other instances, methods, apparatuses, systems, etc., that would be known by one of ordinary skill have not been described in detail so as not to obscure claimed subject matter. Some portions of the detailed description have been presented in terms of algorithms or symbolic representations of operations on data bits or binary digital signals stored within a computing system memory, such as a computer memory. These algorithmic descriptions or representations are examples of techniques used by those of ordinary skill in the data processing arts to convey the substance of their work to others skilled in the art. An algorithm as described herein, and generally, is considered to be a self-consistent sequence of operations or similar processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has been convenient at times, principally for reasons of common usage, to refer to such signals as bits, data, values, elements, symbols, characters, terms, numbers, numerals or the like. It should be understood, however, that all of these and similar terms are to be associated with appropriate physical quantities and are merely convenient labels. Unless specifically stated otherwise, as apparent from the following discussion, it is appreciated that throughout this specification discussions utilizing terms such as “processing,” “computing,” “calculating,” “determining” or the like refer to actions or processes of a computing platform, such as a computer or a similar electronic computing device, that manipulates or transforms data represented as physical electronic or magnetic quantities within memories, registers, or other information storage devices, transmission devices, or display devices of the computing platform.

While this invention has been particularly shown and described with references to preferred embodiments thereof, it will be understood by those skilled in the art that various changes in form and details may be made therein without departing from the spirit and scope of the present application as defined by the appended claims. Such variations are intended to be covered by the scope of this present application. As such, the foregoing description of embodiments of the present application is not intended to be limiting. Rather, any limitations to the invention are presented in the following claims.

What is claimed is:

1. A hand tool attachment assembly comprising:
a cap, the cap including a hollowed volume to matably secure the cap to a distal end of a respective hand tool;
an outer tube affixed to the cap opposite the hollowed volume; and
an inner tube disposed in the outer tube, the inner tube rotatable within the outer tube.

2. The hand tool attachment assembly as in claim 1, wherein a distal end of the inner tube includes a blade; and
wherein a distal end of the outer tube includes a cutting window, the blade of the inner tube disposed to rotate in a portion of the outer tube including the cutting window.

3. The hand tool attachment assembly as in claim 2, wherein the inner tube is longer than the outer tube; and
wherein the outer tube includes a stop preventing the inner tube from sliding beyond the window at the distal end of the outer tube.

4. The hand tool attachment assembly as in claim 3, wherein the inner tube is slidably removable from the outer tube.

5. The hand tool attachment assembly as in claim 4, wherein a proximal end of the inner tube extends into the hollowed volume of the cap.

6. The hand tool attachment assembly as in claim 5, wherein the proximal end of the inner tube includes a keyed interface for coupling to a distal end of a drive assembly in a cavity of the respective hand tool.

7. The hand tool attachment assembly as in claim 6, wherein the hollowed volume in the cap creates an air-tight connection and forms part of a cavity in the distal end of the respective hand tool.

8. The hand tool attachment assembly as in claim 7 further comprising:
a fastening resource disposed on an inner surface of the cap, the fastening resource securedly attaching the cap to the distal end of the respective hand tool.

9. The hand tool attachment assembly as in claim 1, wherein the inner tube is slidably removable from the outer tube through the hollowed volume of the cap.

10. The hand tool attachment assembly as in claim 10, wherein the outer tube includes a stop preventing the inner tube from sliding beyond a window at the distal end of the outer tube; and
wherein a proximal end of the outer tube is secured to an opening in the cap, the inner tube slidably removable from the outer tube and the opening in the cap.

11. A method of fabricating a blade assembly, the method comprising:
receiving a cap, the cap including a hollowed volume to matably secure the cap to a distal end of a respective hand tool;
receiving a first tube, the first tube including a cutting window disposed at a distal end opposite the cap;
affixing the first tube to the cap opposite the hollowed volume; and
inserting a second tube in the first tube, the second tube rotatable within the first tube.

12. The method as in claim 11 further comprising:
fabricating a distal end of the second tube to include a blade, the blade of the second tube disposed to rotate in a portion of the first tube including the cutting window.

13. The method as in claim 12 further comprising:
fabricating the first tube to include a stop preventing the second tube from sliding beyond the window at the distal end of the first tube.

14. The method as in claim 13, wherein the second tube is slidably removable from the first tube.

15. The method as in claim 14, wherein a portion of the second tube extends into the hollowed volume of the cap.

16. The method as in claim 15 further comprising:
producing the proximal end of the second tube to include a keyed interface for coupling to a distal end of a drive assembly in a cavity of the respective hand tool.

17. A method comprising:
receiving a hand tool, a distal end of the hand tool opposite a handle of the hand tool including an opening and a hollowed volume, the hollowed volume including a rotatable drive adapter;
receiving a blade assembly, the blade assembly including an inner tube disposed in an outer tube extending axially from a cap of the blade assembly;
engaging an end of the inner tube passing through the cap into the rotatable drive adapter; and
securing the cap of the blade assembly to the distal end of the hand tool.

18. The method as in claim 17, wherein securing the cap blade assembly to the distal end of the hand tool causes the hollowed volume to become a fluid-tight cavity, the cap covering the opening.

19. The method as in claim 18 further comprising:
operating a drive control resource of the hand tool to rotate the rotatable drive adapter, the rotatable drive adapter rotating the inner tube disposed within the outer tube.

20. The method as in claim 19 further comprising:
applying suction to the fluid-tight cavity, application of the suction to the fluid-tight cavity drawing matter through the inner tube and a hollowed volume of the rotatable drive adapter out a respective port of the fluid-tight cavity.

21. The method as in claim 17, wherein the rotatable drive adapter is spring-loaded; and
wherein securing the cap of the blade assembly to the distal end of the hand tool causes the spring-loaded rotatable drive adapter to exert an axial force on the inner tube towards a cutting window on a distal end of the outer tube, the distal end of the outer tube including a stop preventing the inner tube from sliding beyond the cutting window.

22. The method as in claim 17 further comprising:
subsequent to engaging and securing:
removing the cap from the distal end of the hand tool; and
exerting a force on the inner tube to remove the inner tube from the outer tube.
